(19) United States
(12) Patent Application Publication
(10) Pub. No.: US 2012/0029542 A1
(43) Pub. Date: Feb. 2, 2012

(54) MICRO-SCALPEL USED TO TREAT CARPAL TUNNEL SYNDROME

(76) Inventor: Chi-Yuen Huang, Changhua City (TW)

(21) Appl. No.: 12/845,732

(22) Filed: Jul. 29, 2010

(51) Int. Cl. A61B 17/3211

(52) U.S. Cl. 606/167

(57) ABSTRACT

A micro-scalpel used to treat carpal tunnel syndrome has an elongated columnar blade, one end of the blade obliquely cut to form a tip, the obliquely-cut surface of the tip sufficiently sharp to pierce and cut skin of a patient. The other end of the blade is connected to a hollow sleeve connector, and the hollow sleeve connector is combined with a handle. The micro-scalpel is oriented perpendicular to a transverse carpal ligament, and swings from its proximal end toward its distal end to cut the transverse carpal ligament, thereby reducing the number of cuts of the blade and saving time for completing the carpal tunnel surgery.

### BACKGROUND OF THE INVENTION

1. Field of the Invention

The present invention relates to a micro-scalpel, and more particularly to a micro-scalpel for treating carpal tunnel syndrome that is capable of cutting the transverse carpal ligament through a very small opening incision.

2. Description of the Related Art

Carpal tunnel syndrome is a very common problem with associated symptoms, which is caused by compression of the median nerve travelling through the carpal tunnel. Carpal tunnel syndrome affects the hands and is an upper limb neuropathy that results in motor and sensory disturbance of the median nerve.

This condition affects individuals by causing pain, paresthesias, and sometimes weakness in the median nerve distribution. Those diagnosed with carpal tunnel syndrome may experience pain, numbness and tingling sensations in the arm, which may extend to the shoulder and neck area; these feelings are more prevalent at night due to various sleeping positions. To aid in the prevention of carpal tunnel syndrome, stretching exercises of the wrist, hand, and fingers have been used to combat the pain and numbness caused by repetitive actions. Other than using recommended stretches and exercises, useful treatments for carpal tunnel syndrome include use of night splints, corticosteroid injections and ultimately surgery.

In carpal tunnel release surgery, the goal is to divide the transverse carpal ligament in two. This is a wide ligament that runs across the hand, from the scaphoid bone to the hamate bone and pisiform. It forms the roof of the carpal tunnel, and when the surgeon cuts across it (i.e., in a line with the ring finger) it no longer presses down on the nerve inside, relieving the pressure. Endoscopic carpal tunnel release is an increasingly popular treatment. Endoscopic carpal tunnel release employs a synovial elevator, probes, knives, and an endoscope that is used to visualize the underside of the transverse carpal ligament.

However, the typical surgery scalpel has the following drawbacks:

1. The surgery requires two cuts to permit entry of the scalpel, which has a hook blade, leading to longer surgical times and larger wounds.

2. The scalpel with a hook blade needs to cut in from the side of patient's palm, and as it is difficult to know the position and thickness of the transverse carpal ligament, it is very possible to accidently sever other nerves or blood vessels during the surgery.

3. The surgical wound is deep and large, which requires more stitches, and peripheral tissue may be damaged as well, causing longer recovery times.

4. Since the surgical wound is relatively large, the entirety of the patient's hand needs to be anesthetized and subject to a tourniquet, which is a complicated process that may lead to intense pain afterwards.

5. Since the surgical wound is deep and large, the overall recovery time for the surgery is longer.

6. After healing, a scar is present on the patient's hand that may be painful.

Therefore, it is desirable to provide a micro-scalpel that is used to treat carpal tunnel syndrome to mitigate and/or obviate the aforementioned problems.

### SUMMARY OF THE INVENTION

An objective of the present invention is to provide a micro-scalpel that is used to treat carpal tunnel syndrome.

In order to achieve the above-mentioned objectives, a micro-scalpel used to treat carpal tunnel syndrome comprises an elongated columnar blade, one end of the blade obliquely cut to form a tip, the obliquely-cut surface of the tip sufficiently sharp to pierce and cut skin of a patient. The other end of the blade is connected to a hollow sleeve connector, and the hollow sleeve connector is combined with a handle When the micro-scalpel is oriented perpendicular to a transverse carpal ligament, and swings from its proximal end toward its distal end to cut the transverse carpal ligament, thereby reducing the number of cutting times of the blade and saving time for completing the carpal tunnel surgery.

With the above-mentioned embodiments, the following benefits can be obtained: 1. After the micro-scalpel pierces the skin of the wrist of the patient, it can also cut the tissue into pieces, which simplifies the surgical procedure and reduces the costs associated with different surgical tools. 2. The wound caused by the tip of the micro-scalpel is very small, and so there is no need for large area stitches; consequently, the healing time is shortened and scarring is reduced or possibly eliminated. 3. Since the tip creates a very small wound, only require local anesthesia is required, which leads to significantly less discomfort for the patient. 4. The micro-scalpel can start at a position at which no nerves or blood vessels are present, the scale on the micro-scalpel indicates the depth of the cut, and the endoscope can be connected to observe and record the surgery to minimize the possibility of damaging other nerves or blood vessels.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view of an embodiment of the present invention.

FIG. 2 is a perspective exploded view of an embodiment of the present invention.

FIG. 3 is a local detailed view of a micro-scalpel according to an embodiment of the present invention.

FIG. 4 is another local detailed view of a micro-scalpel according to an embodiment of the present invention.

FIG. 5 is a schematic drawing of operating a micro-scalpel according to an embodiment of the present invention.

FIG. 6 is a cross-sectional view of operating a micro-scalpel according to an embodiment of the present invention.

FIG. 7 is another cross-sectional view of operating a micro-scalpel according to an embodiment of the present invention.

FIG. 8 is a perspective exploded view of another embodiment of the present invention.

FIG. 9 is a schematic drawing of operating the micro-scalpel according to another embodiment of the present invention.

FIG. 10 is a schematic drawing of another embodiment of the present invention.

FIG. 11 is a schematic drawing of an embodiment of the present invention being connected to an endoscope.

### DETAILED DESCRIPTION OF THE PREFERRED EMBODIMENT

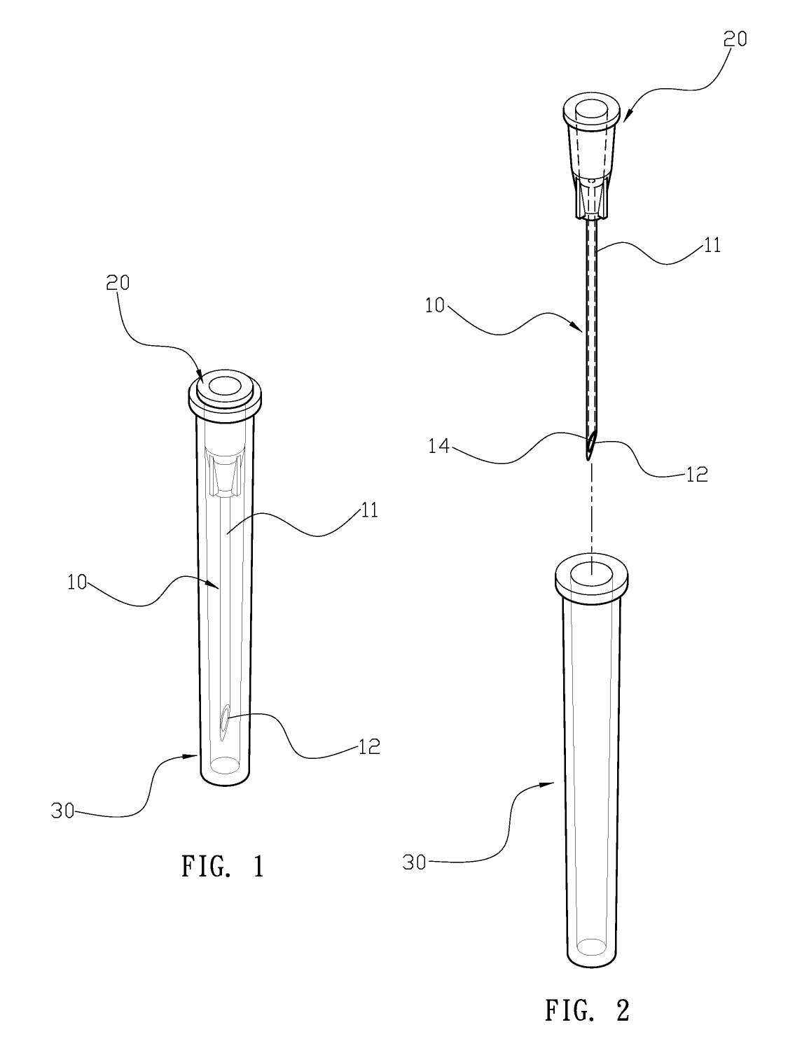

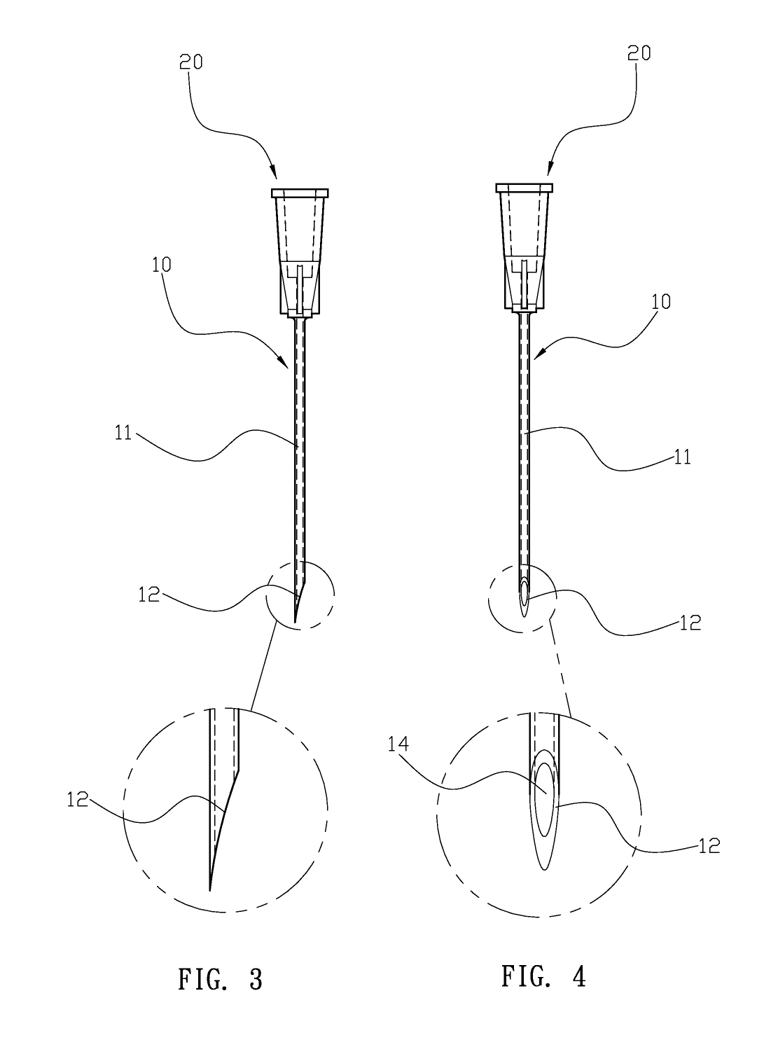

First, please refer to FIG. 1 to FIG. 4. A micro-scalpel 10 has an elongated columnar blade 11, with one end of the blade 11 obliquely cut to form a tip 12, and the other end of the blade is connected to a hollow sleeve connector 20. The obliquely-cut surface of the tip 12 of the micro-scalpel 10 is curved and thin to thereby make the periphery of the tip 12 sharper, such that the obliquely-cut surface of the tip is sufficiently sharp to pierce and cut the skin of a patient. Furthermore, a protection cover may jacket the outside the blade 11, and the protection cover may extend to the tip 12 to protect a user from getting hurt by the tip 12.

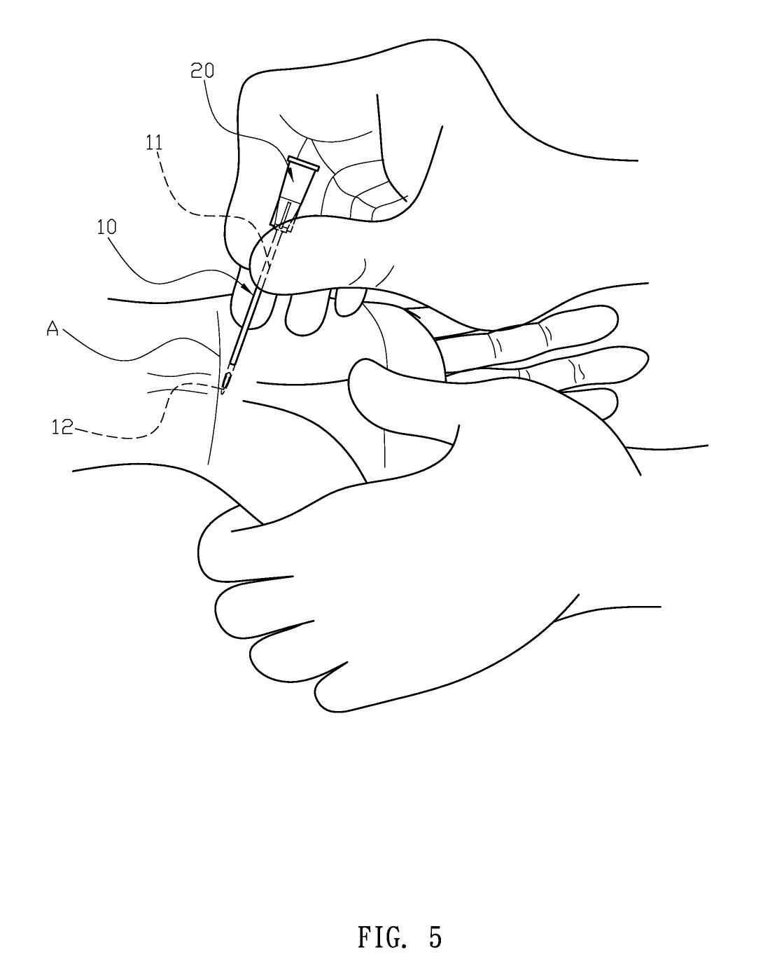

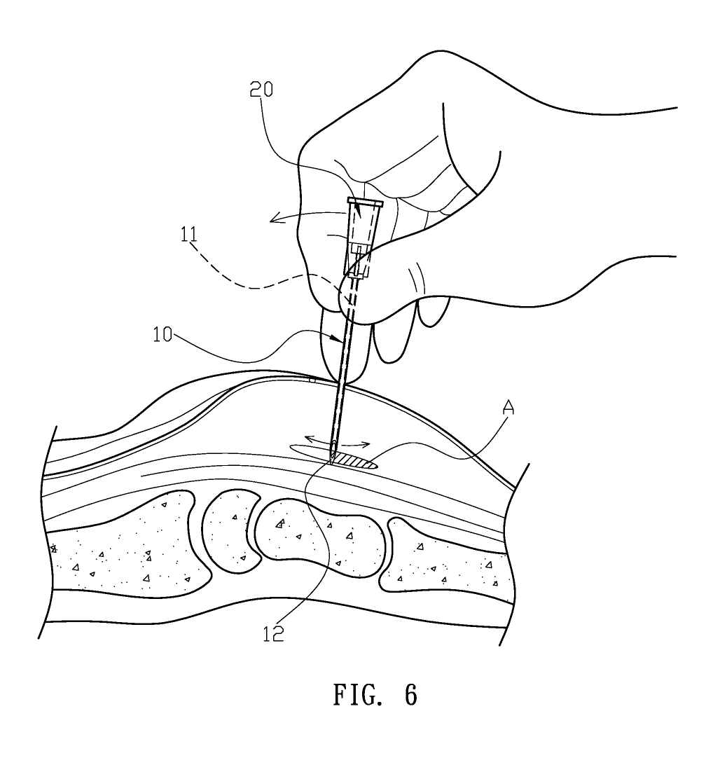

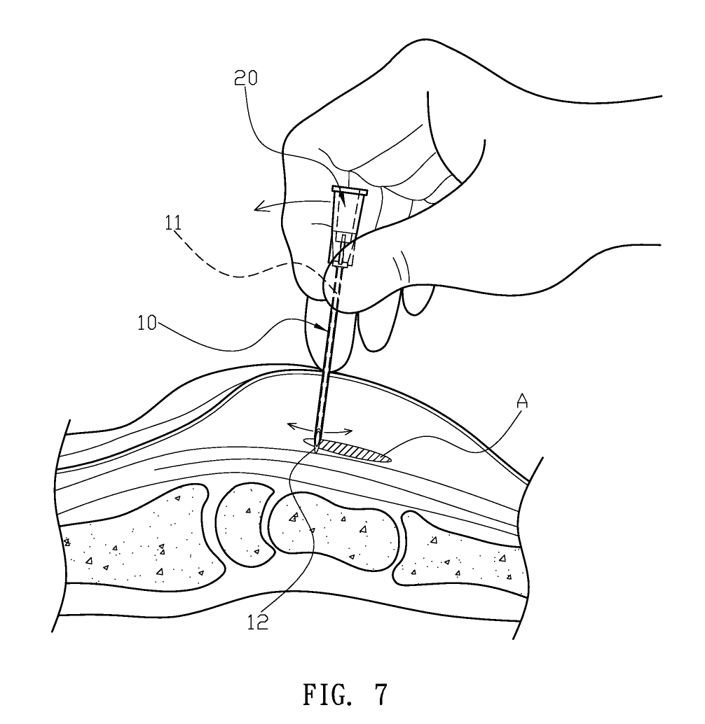

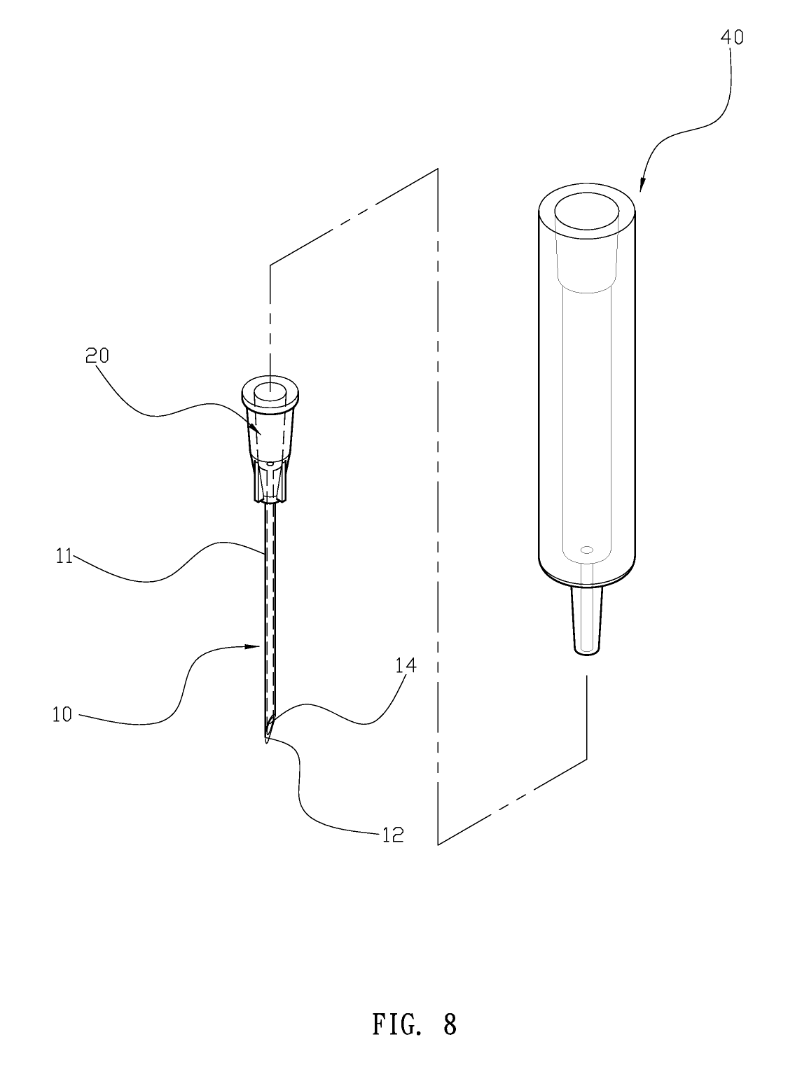

Please refer to FIG. 5 and FIG. 6. In use, the tip 12 of the micro-scalpel 10 is pressed down to pierce the skin of the wrist of the patient suffering from carpal tunnel syndrome; the micro-scalpel 10 is oriented perpendicular to the transverse carpal ligament and the micro-scalpel is swung from the proximal end of the ligament toward the distal end to cut the transverse carpal ligament; consequently the number of cuts is reduced and the time for completing the carpal tunnel surgery is also shortened. Furthermore, since the tip 12 creates a very small opening cut, only local anesthesia is required and the incision does not require a large number of stitches. As a result, the surgical procedure can be shortened and the pain experienced by the patient is reduced. During the surgery, the transverse carpal ligament A is cut at two different points, as shown in FIG. 7, for a more detailed view, which prevents accidental damage to other nerves or veins for better surgical results.

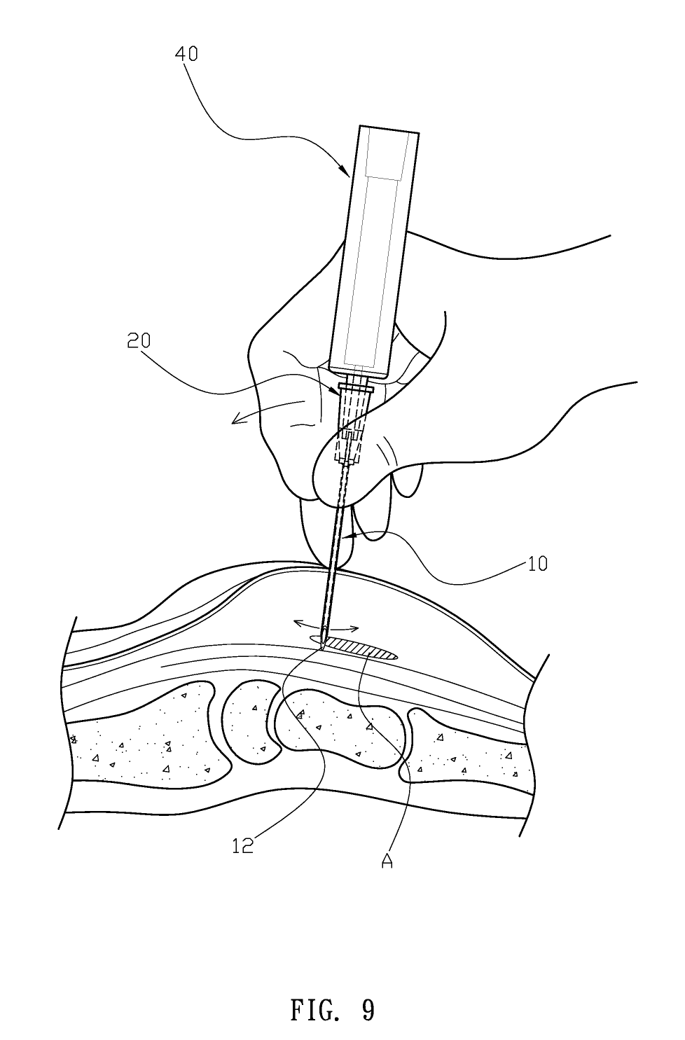

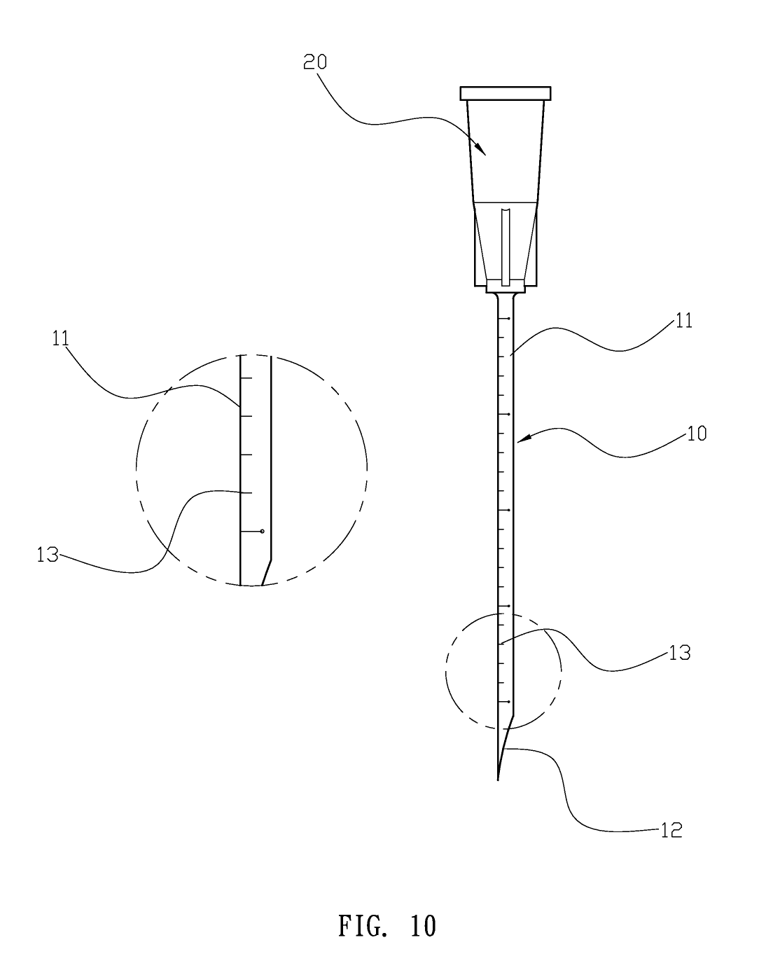

Please refer to FIG. 8 and FIG. 9. The micro-scalpel 10 can also have a handle 40 connected to the hollow sleeve connector 20 for better holding stability. Furthermore, the handle 40 may be monolithically formed with the sleeve connector 20, or the handle 40 may be threadedly connected to the sleeve connector 20. The outer surface of the blade 11 of the micro-scalpel 10 may be provided with a scale 13 having different colors, as shown in FIG. 10. The scale 13 can indicate the insertion depth of the micro-scalpel 10 during surgery.

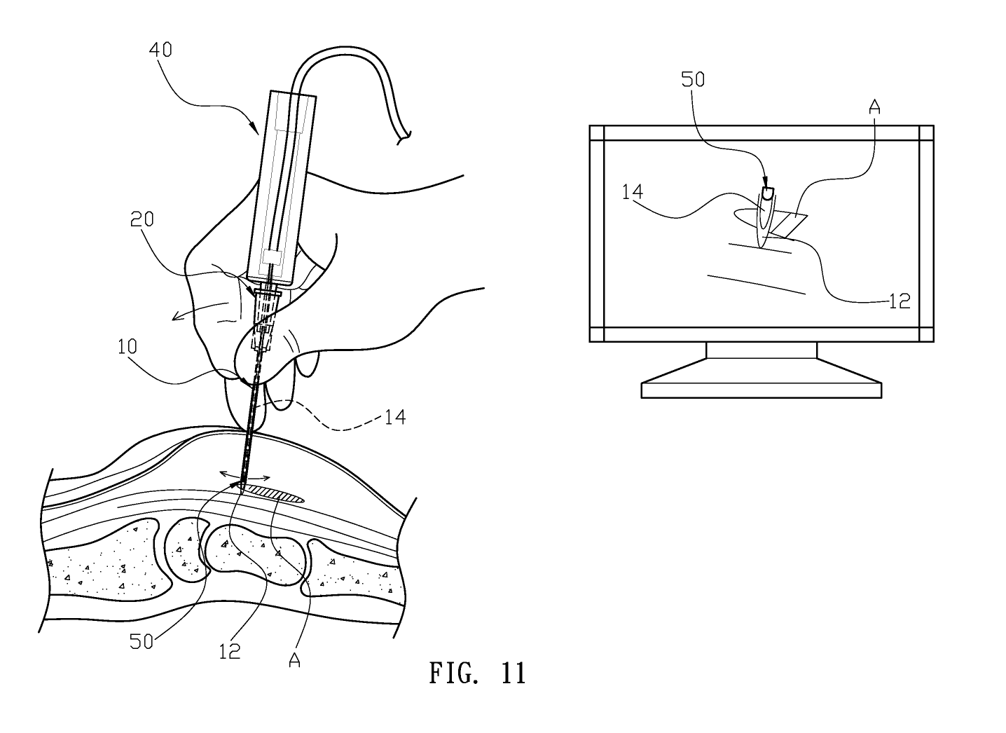

The blade 11 of the micro-scalpel 10 may be solid or may have a hollow through-hole 14. The hollow through-hole 14 can not only be used for releasing blood but may also provide a space for an endoscope 50 that employs one or more optical fibers, thereby permitting observing and recording of the surgery, as shown in FIG. 11.

With the above-mentioned embodiments, the following benefits can be obtained: 1. After the micro-scalpel 10 pierces the skin of the wrist of the patient, it can also cut the tissue into pieces, which simplifies the surgical procedure and reduces the costs associated with different surgical tools. 2. The wound caused by the tip 12 of the micro-scalpel 10 is very small, and so there is no need for large area stitches; consequently, the healing time is shortened and scarring is reduced or possibly eliminated. 3. Since the tip 12 creates a very small wound, only require local anesthesia is required, which leads to significantly less discomfort for the patient. 4. The micro-scalpel 10 can start at a position at which no nerves or blood vessels are present, the scale 13 on the micro-scalpel 10 indicates the depth of the cut, and the endoscope 50 can be connected to observe and record the surgery to minimize the possibility of damaging other nerves or blood vessels.

Although the present invention has been explained in relation to its preferred embodiment, it is to be understood that many other possible modifications and variations can be made without departing from the spirit and scope of the invention as hereinafter claimed.

What is claimed is:

1. A micro-scalpel used to treat carpal tunnel syndrome, characterized in that: the micro-scalpel has an elongated columnar blade, one end of the blade obliquely cut to form a tip, the obliquely-cut surface of the tip sufficiently sharp to pierce and cut skin of a patient.

2. A method of treating a patient suffering from carpal tunnel syndrome by employing the micro-scalpel of claim 1, the method comprising:
causing the tip of the micro-scalpel to pierce the skin of the wrist of the patient suffering from carpal tunnel syndrome at a position at which no nerves or blood vessels are present, the micro-scalpel oriented substantially perpendicular to a transverse carpal ligament, and swinging the micro-scalpel from a proximal end of the transverse carpal ligament toward a distal end of the transverse carpal ligament to cut the transverse carpal ligament, thereby reducing the number of cutting times of the blade and saving time for completing the carpal tunnel surgery.

3. The micro-scalpel according to claim 1, wherein the other end of the blade is connected to a hollow sleeve connector, and the hollow sleeve connector is combined with a handle.

4. The micro-scalpel according to claim 3, wherein the handle is monolithically formed with the sleeve connector.

5. The micro-scalpel according to claim 3, wherein the handle is threadedly connected to the sleeve connector.

6. The micro-scalpel according to claim 1, wherein the obliquely-cut surface of the tip of the micro-scalpel is formed curved and thin to thereby make the periphery of the tip sharper.

7. The micro-scalpel according to claim 1, wherein a protection cover is jacketed outside the blade, the protection cover extending to the tip for protecting a user from getting hurt by the tip.

8. The micro-scalpel according to claim 1, wherein the outer surface of the blade of the micro-scalpel is provided with a scale having different colors.

9. The micro-scalpel according to claim 1, wherein the blade of the micro-scalpel is solid.

10. The micro-scalpel according to claim 1, wherein the blade of the micro-scalpel is provided with a hollow through-hole.

11. The micro-scalpel according to claim 10, wherein an endoscope having an optical fiber is disposed through the hollow through-hole.
